(19) United States
(12) Patent Application Publication
(10) Pub. No.: US 2022/0101961 A1
(43) Pub. Date: Mar. 31, 2022

(54) SYSTEMS AND METHODS FOR MATCHING MEDICAL RECORDS FOR PATIENTS ACROSS DISPARATE MEDICAL PROVIDERS TO FACILITATE CONTINUITY OF CARE

(71) Applicant: MEDICOM TECHNOLOGIES INC., RALEIGH, NC (US)

(72) Inventors: CHASE BALLARD, RALEIGH, NC (US); BRENT GOLDSTEIN, RALEIGH, NC (US); TOMMY LUO, RALEIGH, NC (US); CHRIS WOODLIEF, RALEIGH, NC (US); MICHAEL ROSENBERG, RALEIGH, NC (US)

(21) Appl. No.: 17/033,379

(22) Filed: Sep. 25, 2020

(51) Int. Cl. G16H 10/60; G16H 15/00; G16H 30/20; G06F 40/205

(52) U.S. Cl. CPC G16H 10/60; G06F 40/205; G16H 30/20; G16H 15/00

(57) ABSTRACT

The present invention relates generally to an automated patient matching system that allows medical records for a patient to be reconciled between disparate medical providers by comparison of inbound medical records to patient demographic information stored by a medical provider. The present invention facilitates the preservation of the continuum of care, and allows inbound medical records to automatically be reconciled and ingested for patients ahead of scheduled appointments or procedures.

### CROSS-REFERENCE TO RELATED APPLICATION

This application claims the benefit of U.S. Provisional Patent Application No. 62/866,668 entitled “SYSTEM AND METHOD FOR MATCHING MEDICAL RECORDS ACROSS DISPARATE DATABASES TO FACILITATE CONTINUITY OF CARE” filed on Jun. 26, 2019, and which is commonly owned, the disclosure of which is incorporated herein by reference in its entirety.

### BACKGROUND

### Field of the Invention

The present invention relates, in general, to systems and methods for automated matching of medical records for patients that reside in disparate databases, such as databases associated with different medical providers, in order to correctly link records associated with the same patient.

### Description of Related Art

It is common for patients to receive medical treatment and care from different providers who may not be affiliated or part of the same health care system, entity or organization. Typically, medical providers employ independent and isolated medical records systems, where each patient is assigned a unique medical record number (“MRN”). Each medical record, which can consist of, for example, patient records, medical images, visit histories, and all other relevant medical and health data for the patient, can be linked to a patient's unique MRN for a specific medical provider.

In a situation where a patient visits a new medical provider, this new medical provider may request the patient's medical records to be sent from a prior medical provider. Upon receiving the external medical record from the prior medical provider, the new medical provider typically must reconcile the external medical record due to inconsistencies and differences that may exist in various patient demographic information between the two medical records, such as for example names, addresses, and the like, as well as differences in the MRN.

Regarding MRN reconciliation, different, unaffiliated medical providers tend to have differing MRN numbering and/or classification schemes which are not universal or consistent with MRN numbering schemes used by other medical providers. In order to ingest an external medical record, the new medical provider must update an existing MRN from a prior medical provider to be consistent and compatible with the new medical provider's medical records system. This reconciliation can be a laborious and time-consuming effort, especially with medical practices that routinely receive a large volume of external medical records.

In addition, traditional patient matching systems that attempt to link medical records located across disparate medical providers tend to utilize patient demographic information or MRN data from a PACS or DICOM storage archive. These types of archives typically implement mechanisms to detect duplicate and orphaned medical records, and as such, medical records staff are more likely to correct such issues in the EHR rather than within a PACS archive, for example.

In addition, medical records can oftentimes be inaccurate due to human entry error. Thus, a mistake in the entry of a medical record may cause traditional patient matching systems to overlook, miss, or incorrectly retrieve medical records, due to, for example, a misspelling of a patient's name, or transposing of numbers in a date of birth or social security number. Traditional patient matching systems do not provide for a detection of such errors, much less a safeguard that requires manual review before such external medical records are ingested by a medical provider.

Therefore, there is a need for an automated patient matching system which allows medical records for a patient to be transferred between different medical providers, where the medical records are automatically reconciled for the receiving medical provider, and where manual intervention safeguards are incorporated to prevent erroneous linking of medical records. As such, the present invention ultimately facilitates the preservation of the continuum of care, and allows medical records to automatically be reconciled for patients ahead of scheduled appointments or procedures.

### SUMMARY

In one embodiment, the invention relates to a method for automated matching of disparate medical records for a patient, comprising: receiving appointment information for the patient by a server; extracting, by the server, a patient attribute and a medical record identifier from the appointment information; storing the patient attribute in a database coupled to the server; receiving, by the server, an inbound medical record, wherein the inbound medical record contains an inbound patient attribute and an inbound medical record identifier; and determining, by the server, if the inbound patient attribute matches the patient attribute, wherein the server is configured to update the inbound medical record identifier to match the medical record identifier if the inbound patient attribute matches the patient attribute.

In another embodiment, the invention relates to a method for automated matching of disparate medical records, comprising: receiving, by a server, an inbound medical record from a remote database, wherein the inbound medical record includes demographic data and an identification number; comparing, by the server, the demographic data in the inbound medical record to stored demographic data in a stored medical record located on a local database, wherein the stored medical record includes a stored medical record identification number, and wherein the local database is communicatively coupled to the server; and updating, by the server, the identification number to match the stored medical record identification number if the demographic data matches the stored demographic data.

In yet another embodiment, the invention relates to a system for automated matching of disparate medical records, comprising: a server coupled to a medical provider; a virtual service coupled to the medical provider, wherein the virtual service is an instance of the server; a patient scheduling system coupled to the virtual service via a data exchange interface, wherein the virtual service is configured to receive appointment information from the patient scheduling system; a parsing engine coupled to the virtual service, wherein the virtual service is configured to extract patient demographic information from the appointment information using the parsing engine; and a database coupled to the virtual service, wherein the virtual service is configured to store the extracted patient demographic information on the database, wherein the virtual service is further configured to compare an inbound medical record to the extracted patient demographic information.

### BRIEF DESCRIPTION OF THE DRAWINGS

These and other embodiments of the disclosure will be discussed with reference to the following exemplary and non-limiting illustrations, in which like elements are numbered similarly, and where:

FIG. 1 is a network architecture diagram of a system of automated patient matching of medical records that reside in disparate databases, according to an embodiment of the invention;

FIG. 2 is a flowchart illustrating the steps of initiating automated patient matching of medical records upon receipt of inbound medical records from an external medical provider, according to an embodiment of the invention;

FIG. 3 is a flowchart illustrating the steps of performing a search of local medical records against an inbound medical record, according to an embodiment of the invention;

FIG. 4 is a flowchart illustrating the steps of performing a search of local medical records against an inbound medical record if a flag is encountered, according to an embodiment of the invention;

FIG. 5 is a flowchart illustrating the steps of ingesting an inbound medical record based on a manual review, according to an embodiment of the invention;

FIG. 6 is a flowchart illustrating the steps of creating a patient matching configuration file, according to an embodiment of the invention;

FIG. 7 is a flowchart illustrating the steps of retrieving patient demographic information from an appointment order for an advanced patient matching process, according to an embodiment of the invention; and

FIG. 8 is a block diagram illustrating a local database coupled to a virtual service, according to an embodiment of the invention.

### DEFINITIONS

The following definitions are meant to aid in the description and understanding of the defined terms in the context of the present invention. The definitions are not meant to limit these terms to less than is described throughout this application. Such definitions are meant to encompass grammatical equivalents.

As used herein, the terms “medical record” and “medical records” can refer to, for example, health and healthcare related data, patient data, patient records and charts, diagnostic notes, opinions, reads and reports, medical test and laboratory results, surgical history, family history, medications, medical allergies, social history, habits (such as, for example, drug, tobacco and alcohol use), immunization history, clinical information, growth and development history, medical encounters, physical examination observations, progress notes, electronic medical records, medical imaging studies, medical and diagnostic images, diagnostic reports, fitness and activity data, medical reports, patient medical history and demographics, encounter and visit histories, admit/discharge/transfer and patient tracking information, scheduling and referrals, orders and results (measurements, observations, impressions, reports), pharmacy and diet information, and the like.

As used herein, the term “medical provider” can refer to, for example, medical clinics, hospitals, medical providers, health insurance providers, diagnostic sites, imaging sites, data customers, and the like.

As used herein, the term “patient scheduling system” and “patient record system” can refer to, for example, an electronic health record (EHR) system, a master patient index (MPI), an enterprise-wide master patient index (eMPI), an electronic medical record system (EMR), a hospital information system (HIS), a Computerized Physician Order Entry systems (CPOE), and Integrating the Healthcare Enterprise Order Placer (IHE OP), a picture archiving and communication system (PACS), a Radiology Information System (RIS), a medical practice management system, a patient scheduling system, a physician referral system, and the like.

As used herein, the term “data exchange interface” and “data exchange format” can refer to, for example, an interface, standard, specification and/or protocol that allows for disparate systems to share data, including, but not limited to, the Health Level Seven (HL7) standard, the Fast Healthcare Interoperability Resources (FHIR) standard, the HPRIM standard, the Continuity of Card Record (CCR) standard, the Continuity of Care Document (CCD) specification, the xDT data exchange format, the Arden syntax, an application programming interface (API), a Portable Document Format (PDF), and direct access protocols for electronic health records systems, and the like.

As used herein, the term “database” and “databases” can refer to, for example, an organized collection of data, generally stored and accessed electronically from a computer system, and which can be implemented as a data store, an archive, a relational database, a SQL database, an object-oriented database, a centralized database, a distributed database, a cloud-based database, a blockchain-based database stored across a distributed ledger, as well as a PACS database, a EHR database, a EMR database, a HIS database, a RIS database, a Vendor Neutral Archive (VNA), and the like.

As used herein, the term “patient identifier”, “patient identifiers” and “patient information” can refer to, for example, a patient first, middle, and/or last name, date of birth, social security number, demographics, sex, physical address, e-mail address, telephone number, insurance information, primary care physician information, universal identifier, accession number, medical record number, an identification number, a patient number, a chart number, a DICOM series number, and the like.

As used herein, the term “Checkpoint” can refer to, for example, a virtual location where data or records are held for manual review, such as a gatekeeper, an e-mail inbox, a messaging system, a database, and the like, as well as a physical location coupled to a device, such as a printer, facsimile machine, a computing device, and the like.

As used herein, the term “machine learning” can refer to, for example, deep learning, reinforcement learning, neural network computing, artificial intelligence computing, fuzzy logic computing, and the like.

As used herein, the term “string comparison algorithm” can refer to, for example, processes and algorithms that utilize fuzzy matching, fast fuzzy techniques, Expectation-Maximization techniques, Wink-Jaro Distance techniques, talisman techniques, k-means techniques, clustering techniques, and the like.

As used herein, the terms “storage location” and “routing location” can refer to, for example, a location associated with a DICOM, HL7, FHIR, HPRIM, CCR, CCD, xDR, PACS, patient record system, and/or patient scheduling system storage, database, repository, or archive.

As used herein, the term “metadata”, “header” and “headers” can refer to, for example, information, data, descriptors located within various file formats, including, but not limited to, the DICOM, NIfTI, MINC, Analyze, JPEG, JPG, TIF, GIF, PNG, and PDF file formats.

### DETAILED DESCRIPTION

It should be understood that aspects of the invention are described herein with reference to the figures, which show illustrative embodiments. The illustrative embodiments herein are not necessarily intended to show all embodiments in accordance with the invention, but rather are used to describe a few illustrative embodiments. Thus, aspects of the invention are not intended to be construed narrowly in view of the illustrative embodiments. In addition, although the invention is described with respect to its application for the transfer of medical information containing DICOM data applications, it is understood that the system could be implemented in any setting where the transfer of any type or form of medical or patient data may be useful. In addition, while use of DICOM data is disclosed herein as a preferred embodiment, the present invention is not limited to the use of DICOM medical images and data, and various data, compression, and imaging formats and standards can be utilized within the scope of this invention. Such formats include, but are not limited to, NIfTI, MINC, and Analyze.

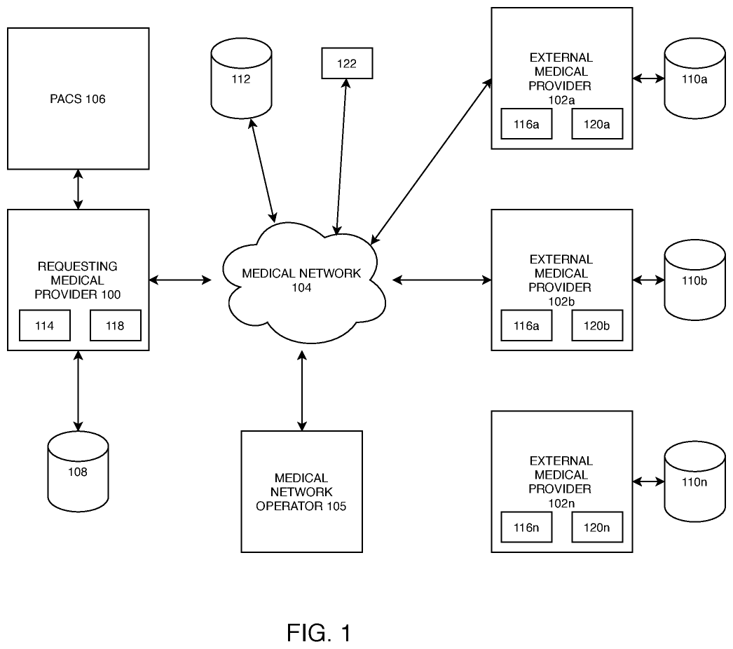

FIG. 1 is a network architecture diagram of a system of automated matching of medical records that reside in disparate databases, according to an embodiment of the invention. In an embodiment, a medical provider 100 is communicatively coupled to at least external medical provider 102 via a medical network 104. The medical network 104 can be a restricted network operated by a medical network operator 105, such that the medical providers 100, 102 are registered with the medical network 104, and have a relationship with the medical network operator 105.

In an embodiment, the medical network 104 includes computing hardware, software, and/or services that facilitate the automated patient matching of medical records associated with the external medical providers 102 with medical records associated with a medical provider 100. In an embodiment, the medical network 104 is configured as a server, virtual server, or a distributed server that can store data, as well as execute programs, algorithms, scripts, and applications.

In an embodiment, the medical network 104 is a proprietary network that facilitates direct peer-to-peer data transfer between the medical providers 100, 102 as described in more detail in commonly owned application Ser. No. 16/281,409, entitled, METHODS AND SYSTEMS FOR TRANSFERRING SECURE DATA AND FACILITATING NEW CLIENT ACQUISITIONS, the contents of which are hereby incorporated by reference in its entirety.

In an embodiment, the medical provider 100 is, or is coupled to, a medical facility, hospital, outpatient clinic, diagnostic imaging center, diagnostic imaging station, and the like. In an embodiment, the medical provider 100 utilizes a PACS 106. The medical provider 100 can be remote from, or locally coupled to, the PACS 106, and the medical provider 100 can be communicatively coupled to multiple local or distributed PACS (not shown).

In an embodiment, the medical provider 100 include computing hardware and software, and can be coupled to a database 108 that contains medical records. In an embodiment, the database 108 is a PACS databases coupled to the PACS 106.

Similarly, each external medical provider 102a-n can include computing hardware and software, and each can be coupled to a respective database 110a-n, as shown in FIG. 1. In an embodiment, the medical network operator 105 can be coupled to a database 112.

In an embodiment, the database 112 can be configured to store replicated versions of data stored on the databases 110a-n. The database 112 can be updated or refreshed to reflect real-time changes to each database 110a-n, or the database 112 can be updated or refreshed on a periodic basis, such as daily, weekly, monthly, etc. In an embodiment, the database 112 can be updated or refreshed based on a triggering event, such as for example, a request for medical records being received from a medical provider 100 by the medical network 104.

Each database 108-112 can be locally stored and managed by its respective associated entity, and each database 108-112 can be distributed or cloud-based, and located remotely from its respective associated entity, such as on a remote server provided by Amazon Web Services® or the like. In an embodiment, each database 108-112 can be a relational database, a SQL database, an object-oriented database, a centralized database, or a distributed database, such as a cloud-based database or a blockchain-based database stored across a distributed ledger. In an embodiment, the databases 108 and 110 are capable of storing data and files in the DICOM format.

In an embodiment, each database 108-112 is located within a LAN, WAN, and/or intranet associated with the respective medical provider 100, 102. In another embodiment, each database 108-112 can be located and outside of a LAN, WAN, and/or intranet associated with the respective medical provider 100, 102.

In an embodiment, each medical provider 100, 102 is configured to run a virtual service 114, 116 that is operated, provided, and/or developed by the medical network operator 105, and are configured as a virtual instance of medical network server 122. In an embodiment, the virtual service 114, 116 can include DICOM standard functionality, and is configured to interface with a patient scheduling system 118, such as, for example, an EHR system. The virtual service 114, 116 is configured to interface with each respective patient scheduling system 118, 120 by, for example, a data exchange interface, such as the HL7 standard, the FHIR standard, the HPRIM standard, the CCR standard, the CCD specification, the xDT data exchange format, the Arden syntax, an API, direct access to an EHR and/or associated databases, and the like.

In an embodiment, the medical network 104 is communicatively coupled to each virtual service 114, 16, and can receive messages, information, and the like from each virtual service 114, 116. For example, the virtual service 114 coupled to the medical provider 100 can be configured to transmit a message to the medical network 104 when a patient appointment is entered into the patient scheduling system 118. In another example, the virtual service 116 coupled to the external medical provider 102 can be configured to transmit a data refresh message to the medical network 104 when database 112 is updated or modified, indicating that the database 112 needs to refresh its data to reflect the updates to database 110.

In an embodiment, each virtual service 114, 116 can function as an edge compute server which allows for distributed processing of data by physically being closer to each respective database 108, 110 and/or patient scheduling system 118, 120.

In another embodiment, the functions of each virtual service 114, 116 can be executed by the medical network 104. In this embodiment, the medical network 104 includes a medical network server 122. In an embodiment, the medical network server 122 can be distributed across multiple computing resources within the medical network 104, or can be a dedicated computing resource.

In another embodiment, instead of a virtual service, each medical provider 100, 102 can include a physical server that locally executes the functions described herein of the virtual service 114, 116.

In an embodiment, medical providers 100, 102 can be communicatively coupled to the medical network 104 via communication links (denoted by arrows in FIG. 1). These communication links may be any type of communication links suitable to allow interaction between the medical providers 100, 102, as well as with the medical network 104 and medical network operator 105. For example, the communication links may each be a wired network, a wireless network, or any combination thereof. Further, communication links may include a distributed computing network, an intranet, a local-area network (LAN) and/or a wide-area network (WAN), or any combination thereof. For example, the LAN may make use of WIFI in its many variations and the WAN may make use of broadband, cellular and/or satellite networks using technologies including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies. However, those of ordinary skill in the art will appreciate that the communication links are not limited thereto. In another embodiment, the communication links may each include ethernet, Firewire, parallel, serial, or USB connections, or short-range communication protocols such as Bluetooth, infrared, Zigbee, and the like.

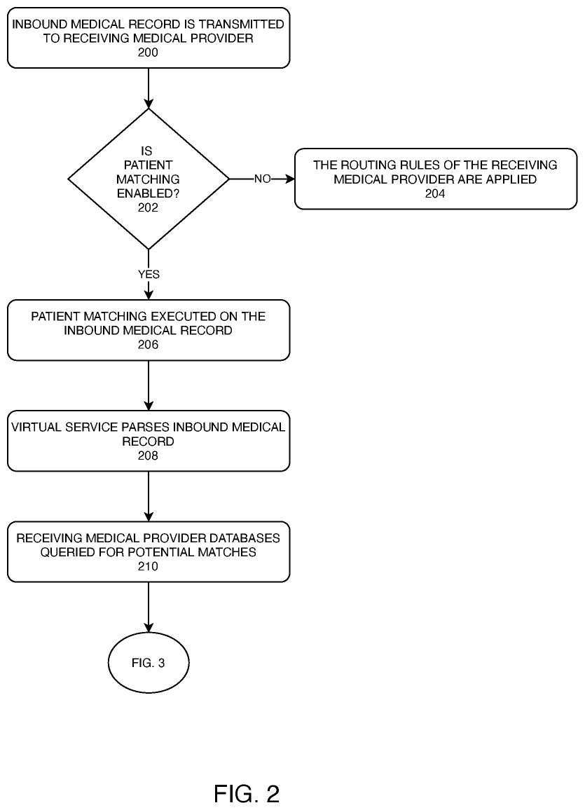

FIG. 2 is a flowchart illustrating the steps of initiating automated patient matching of medical records upon receipt of inbound medical records from an external medical provider, according to an embodiment of the invention. In step 200, an external medical provider 102 transmits an inbound medical record to a medical provider 100 (i.e., a receiving medical provider). In an embodiment, the inbound medical record can originate on a database 110 associated with the external medical provider 102.

In an embodiment, the inbound medical record is transmitted via the medical network 104. In another embodiment, the inbound medical record is transmitted using a direct transfer mechanism, peer-to-peer transfer protocol, a cloud-based transfer mechanism, a remote upload functionality, and the like.

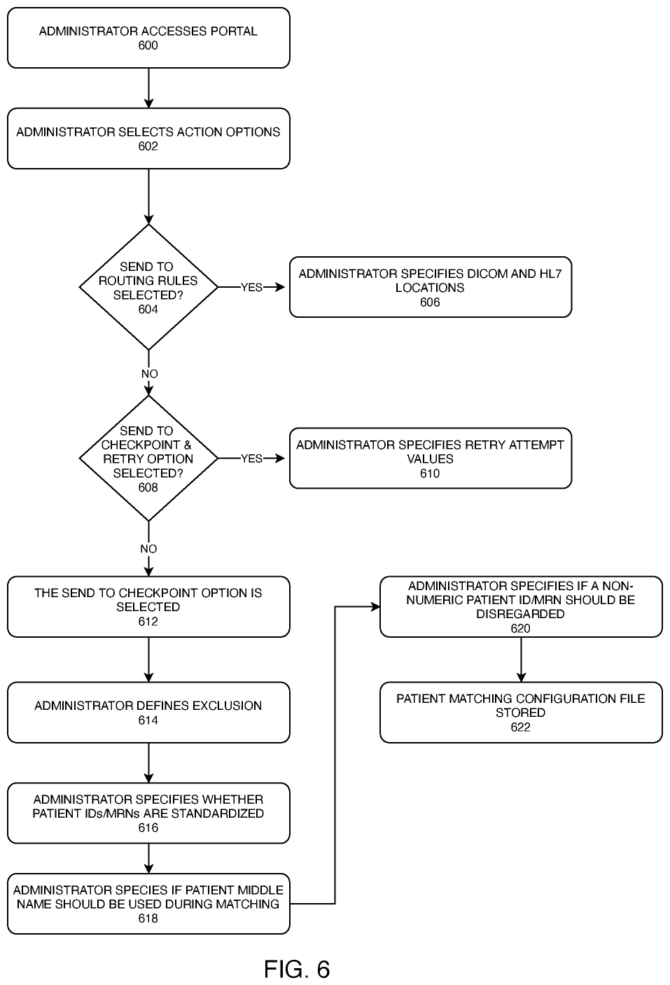

After the inbound medical record is received by the virtual service 114 of the medical provider 100 in step 200, the process continues to step 202, where the virtual service 114 determines if the patient matching function has been enabled by the medical provider 100. If the patient matching function has not been enabled by an administrator associated with the medical provider 100, then the process continues to step 204, where the routing rules of the medical provider 100 are applied to the inbound medical record. The routing rules are defined in a routing rules configuration file, as is described in more detail herein with regards to FIG. 6.

In an embodiment, the patient matching function can be disabled entirely by the medical provider 100, regardless of the source, type, and nature of the inbound medical record, and regardless of the patient that is associated with the inbound medical record. In another embodiment, the patient matching function can be selectively disabled based on patient matching exclusion rules which can be defined during a patient matching configuration process, as is described in more detail herein with regards to FIG. 6. In an embodiment, the exclusion rules can specify a certain time or time period where the patient matching function is enabled or disabled. For example, in a “nighthawk” setting, where imaging studies are read and dictated after hours or overnight, the administrator can choose to disable the patient matching function.

In addition, the administrator can specify certain scenarios where a prefix, suffix, notation, or other identifier is added to an inbound medical record. For example, in a “nighthawk” setting, the patient matching function may be disabled so that inbound medical records are not sent to the Checkpoint, or otherwise held in queue for a manual review, as medical records staff may not be available during overnight hours. During a “nighthawk” setting, the exclusion rules can specify that a prefix is added to a session number (or other identifier) associated with the inbound medical record. The prefix can include, for example, the name of the external medical provider, an alphanumeric character or string (i.e., “NH” or “NightHawk”), and the like. During regular business hours, the medical provider 100 can identify prefixed inbound medical records, and can take further action for reconciliation and matching with internal records.

For example, the patient matching function can be selectively enabled or disabled based on a specific patient or list of patients, a patient demographic (i.e., an age range, gender, etc.), a specific external medical provider or list of external medical providers, a medical imaging modality of the medical record, and the like.

In another embodiment, the patient matching function can be selectively enabled or disabled based on a volume of inbound medical records. For instance, if the virtual service 114 receives more than a threshold number of inbound medical records within a pre-determined period of time, then the virtual service 114 can enable the patient matching function. In an example, if the virtual service 114 receives ten inbound medical records within an hour, then the virtual service 114 can enables patient matching in order to alleviate the amount of manual patient matching operations that must be performed manually by the medical provider 100.

In an embodiment, the threshold number of inbound medical records and the pre-determined period of time can be configured during the patient matching configuration process and stored in a patient matching configuration file, as is described in more detail herein with regards to FIG. 6.

If, however, the patient matching function has been enabled by the medical provider 100, then the process continues to step 206, where the patient matching process is executed on the inbound medical record by the virtual service 114. In an embodiment, the virtual service 114 can parse the metadata of the inbound medical record for comparison against a storage location, or multiple storage locations, associated with the medical provider 100. In a preferred embodiment, the metadata can include DICOM metadata, and the storage location can be a DICOM database location. The DICOM metadata can include, for example, information about the medical image data, such as the size, dimensions, resolution, bit depth, modality used to create the data, series number and description, and equipment settings used to capture the image, as well as patient information, scan data, associated physician information, equipment location information, and the like.

In an embodiment, the virtual server 114 can parse the entire inbound medical record for comparison against a storage location. For example, medical information such as a medical history, clinical data, procedure or treatment history, prescription history, and the like, can be parsed by the virtual server 114 so that the parsed data can be compared against records in the storage location.

In another embodiment, the inbound medical record is in a FHIR format, and an API is utilized to retrieve data from the patient record system coupled to the medical provider 100, such as an EMR or EHR system. The virtual service 114 can compare or cross-reference the retrieved data with the inbound medical record for patient matching purposes.

Next, in step 208, the virtual service 114 parses the inbound medical record to identify and extract various information, such as attributes or fields specified during the patient matching configuration process, and stored in, for example, the patient matching configuration file. For example, the patient matching configuration file can specify that certain patient attributes, such as patient demographic information, be included or excluded for patient matching purposes. This patient demographic information can include, but is not limited to, a patient first name, middle name, middle initial, last name (also known as a family name and surname), date of birth, social security number, sex/gender, address, telephone number, insurance information, primary care physician information, universal identifier, and the like.

The patient matching function offers a higher degree for record linkage compared to deterministic matching since the present invention relies on patient demographic information recorded in, for example, a MPI, eMPI, EHR, RIS, or medical information database. Use of patient demographic information provides a higher accuracy compared to using traditional PACS and DICOM storage archives, as these types of archives typically implement mechanisms to detect duplicate and orphaned medical records, and as such, medical records staff are more likely to correct such issues in the MPI, eMPI, and EHR rather than within a PACS archive, for example. Typically, a PACS archive is not commonly relied upon as a verified source of real-time accurate records, i.e., a source of truth.

In an embodiment, the virtual service 114 includes a parsing engine to analyze text, characters, numbers, and alphanumeric contents located within the data feed. The parsing engine can be configured to accept as an input the inbound patent record, and identify various medical and healthcare related lexicons, such as, for example, patient information, patient demographic information, medical procedure information, billing information, coding data, external medical provider information, referring physician information, and the like.

In an embodiment, the inbound medical record may be in various formats, such as, for example, HL7, FHIR, JSON, XML, RDF, Turtle, PDF and the like. In a preferred embodiment, the inbound medical record is in a HL7 format. The parsing engine may be configured to parse the data feed in the HL7 format, and may be configured to assign a dictionary to the data feed in the HL7 format, so that the inbound medical record may be translated. The parsing engine may be configured to parse data that is formatted in a HPRIM, GMSIH, JAHIS format, or any other format suitable and compliant with Integrating the Healthcare Enterprise (IHE) standards. In addition, parsing engines dedicated to the different versions of HL7 as well as various other medical data standards and/or dedicated to other known formats may be utilized by the parsing engine without departing from the scope of the present invention. In an embodiment, the parsing engine can utilize XML parsing, text-based parsing, and the like.

In an embodiment, the virtual service 114 may include one or more parsing engines, whereby a first parsing engine may be configured to parse any incoming information and/or distribute the incoming data to other parsing engines.

In an embodiment, the parsing engine can utilize a machine learning function or algorithm to parse the inbound medical record, as well as to contribute to a dynamically updated learning dictionary. By using machine learning analysis on information extracted from inbound medical records over time, the parsing engine can be optimized to predictively optimize text and character recognition and analysis functions.

In an embodiment, the machine learning function employs natural language processing (NLP) to parse the inbound medical record. The NPL techniques that can be utilized include, but are not limited to, named entity recognition, text summarization, aspect mining, topic modeling, Latent Semantic Analysis, Probabilistic Latent Semantic Analysis, Latent Dirichlet Allocation, Correlated Topic Model, text embedding, neural machine translation, and the like.

Next, in step 210, the virtual service 114 generates a patient matching query based on the patient matching configuration file. The patient matching query is transmitted to the database 108 by the virtual service 114 in order to determine if there are any medical records stored in the database 108 which match the patient associated with the inbound medical record. In an embodiment, the medical provider 100 includes multiple databases which are queried. Such databases can include, for example, databases associated with an EHR system, a PACS, a VNA, and the like. In an embodiment, the attributes specified in the patient matching configuration file are used to query the database 108.

In an embodiment, all storage locations associated with the medical provider 100 are queried by the virtual service 114 using the parsed information extracted from the inbound medical record.

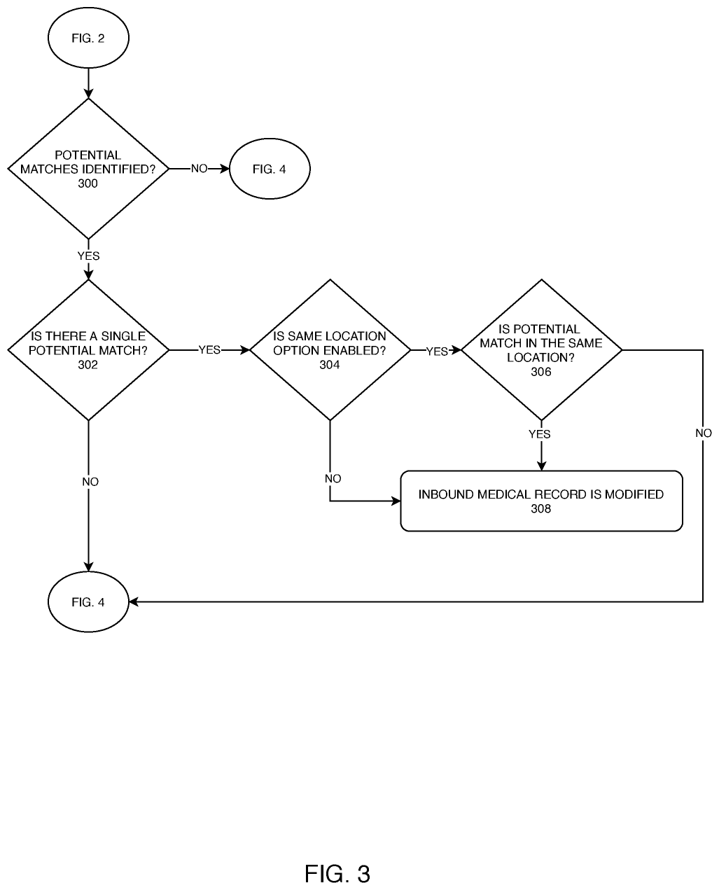

The process then continues to the steps described herein in more detail with reference to FIG. 3. FIG. 3 is a flowchart illustrating the steps of performing a search of local medical records against an inbound medical record, according to an embodiment of the invention.

In step 300, the virtual service 114 transmits the patient matching query to the database 108. In an embodiment, the virtual service 114 utilizes a C-FIND request which conforms to the DICOM standard. The C-FIND service is an operation by which relevant patient information and medical records can be queried across disparate databases. In another embodiment, the virtual service 114 can utilize SQL commands to query the database 108.

The C-FIND request is transmitted to the database 108, or each associated database of the medical provider 100. The C-FIND request can include various identifiers based on the patient matching configuration file.

In an embodiment, the contents of the C-FIND request can be populated with query criteria based on options, rules and/or selections defined in the patient matching configuration file. In an embodiment, the C-FIND request can be populated with default values in the event the patient matching configuration file does not specify query criteria. Such default values can include, for example, the entirety of patient demographic information that is typically included in header fields, such as a first name, a last name, a middle name (and/or middle initial), date of birth, and sex. In an embodiment, the virtual service 114 can utilize a deterministic algorithm to compare the inbound medical record to the records stored in the database 108 in order to identify one-for-one character and/or string matches.

In another embodiment, the virtual service 114 can query the database 108 using the WADO protocol, which can be utilized to obtain medical images stored on database 108.

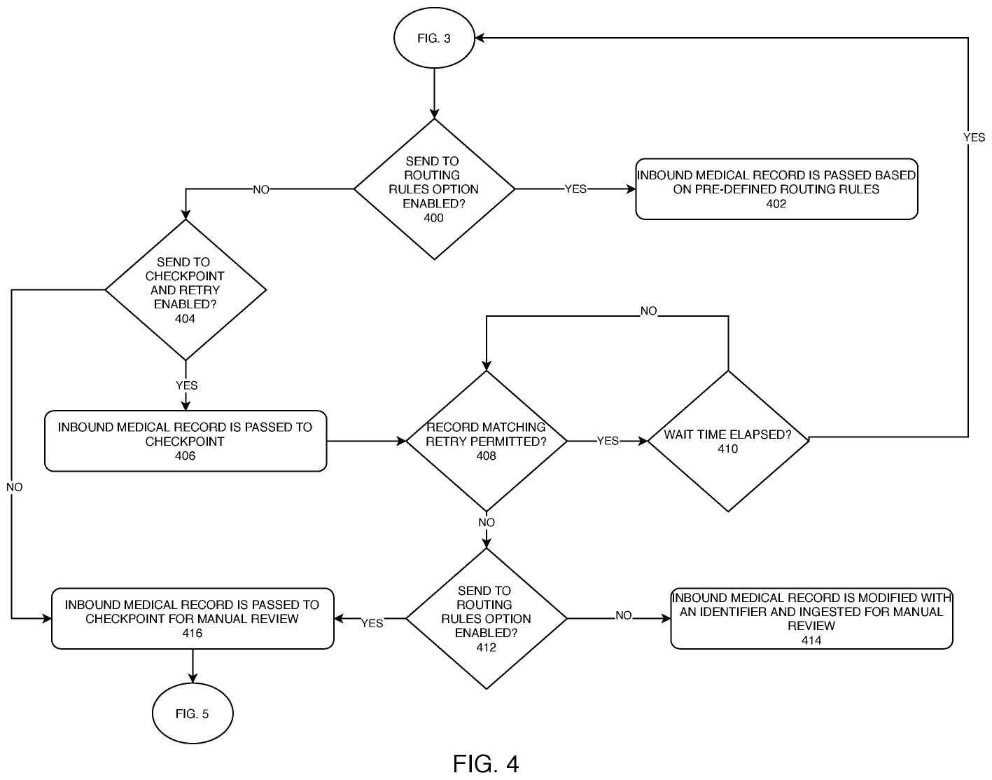

If the patient matching query returns potential matching records from the database 108, then, the process continues to step 302, where the virtual service 114 determines if there is a single potential matching medical record in the database 108 (i.e., a “single match”). However, if the patient matching query does not result in any matching medical records stored on the database 108, then the virtual service 114 applies a flag to the inbound medical record, and the process continues to step 400, as is described in more detail herein with regards to FIG. 4.

A single match is obtained when there is only one unique patient record in the database 108 matching the attributes specified in the patient matching configuration file. In an embodiment, a single match can occur when the one unique patient record in the database 108 contains the same sex, first name, last name, and date of birth as the inbound medical record. For example, a single match is not obtained if the patient matching configuration file specifies a “first name” and “last name” as attributes, and the query results in multiple patient records in the database 108 having identical first and last names. This example is a non-limiting illustration, and not intended to limit the scope of the invention.

In an embodiment, in addition to the sex, first name, last name, and date of birth fields, various other fields can be specified in the patient matching configuration file by the administrator, such as, but not limited to, social security number, demographics, physical address, e-mail address, telephone number, insurance information, primary care physician information, universal identifier, accession number, medical record number, an identification number, a patient number, a chart number, a DICOM series number, and the like. In addition, medical information, such as a medical history, clinical data, procedure or treatment history, prescription history, and the like, can be specified in the patient matching configuration file.

If there is a single potential matching record in the database 108, then the process continues to step 304, where the virtual service 114 determines if the “same location” option has been enabled by the administrator. If the “same location” option has been enabled, then the process continues to step 306, where the virtual service 114 determines if the storage location of the single potential matching record is the same as the destination storage location specified in the patient matching configuration file. In an embodiment, the virtual service 114 can evaluate and compare the two storage locations using, for example, C-FIND requests, SQL queries, or similar commands to determine the contents of each storage location. In an example, the destination storage location can be a radiology PACS, however, the location of the single potential matching record may be a cardiology PACS. In this scenario, the radiology PACS and cardiology PACS may not result in a “same location” match, based on the settings in patient matching configuration file. In this scenario, the medical provider 100 may want to ignore potential matches that do not reside in the destination storage location where inbound medical records are desired to be routed. The use of PACS storage locations in this example is a non-limiting illustration, and not intended to limit the scope of the invention.

However, if the “same location” has not been enabled, then the virtual service 114 applies a flag to the inbound medical record, and the process continues to step 400, as is described in more detail herein with regards to FIG. 4.

If the virtual service 114 determines in step 306 that the single potential matching record and the inbound medical record have the same storage location, then the process continues to step 308, where the inbound medical record is modified. In an embodiment, at least one patient identifier contained in the inbound medical record is modified so that it is consistent with a corresponding patient identifier contained in the single potential matching record.

In an embodiment, the virtual service 114 can update the MRN contained in the inbound medical record to match the MRN in the single potential matching record. In many instances, the medical provider 100 may have an MRN numbering and/or classification scheme that is not consistent with MRN scheme used by the external medical providers 102.

In an embodiment, specified fields in the header of the inbound medical record are modified to match the corresponding fields on the single potential matching record. To accomplish such a modification, a C-STORE command, such as via a dcm4che-storescu utility library, can be utilized. As discussed herein, the inbound medical record may be in a NIfTI, MINC, or Analyze format, and as such, the relevant headers corresponding to those file formats are used to match the corresponding fields on the single potential matching record.

FIG. 4 is a flowchart illustrating the steps of performing a search of local medical records against an inbound medical record if a flag is encountered, according to an embodiment of the invention. In an embodiment, a “flag” refers to a scenario where a single potential matching record is not identified, or if there is not a “same location” match.

In step 400, the virtual service 114 determines if a “send to routing rules” option has been enabled by the administrator. If the “send to routing rules” option has been enabled, then the process continues to step 402, where transformations indicated in the routing rules configuration file are applied to the inbound medical record. The inbound medical record is then ingested by the medical provider 100 into its database 108, and an in embodiment, specifically into a DICOM storage or archive. In another embodiment, the inbound medical record is ingested into a patient scheduling system or patient record system.

In an embodiment, such routing rules can include, for example, passing the inbound medical record for manual review, applying pre-processing to the data, denying receipt of the inbound medical record, and the like.

If, however, the “send to routing rules” option has not been enabled, then in step 404, the virtual service 114 determines if the “send to checkpoint and retry” option has been enabled by the administrator. If the “send to checkpoint and retry” option has been enabled, then the process continues to step 406, where the inbound medical record is passed to a Checkpoint. In an embodiment, the virtual service 114 then attempts to retry the patient matching process in step 408 based on a retry attempt criteria that is defined in the patient matching configuration file. The retry attempt criteria can include, for example, a retry frequency, maximum number of retries that may be attempted, and/or a wait time between retry attempts.

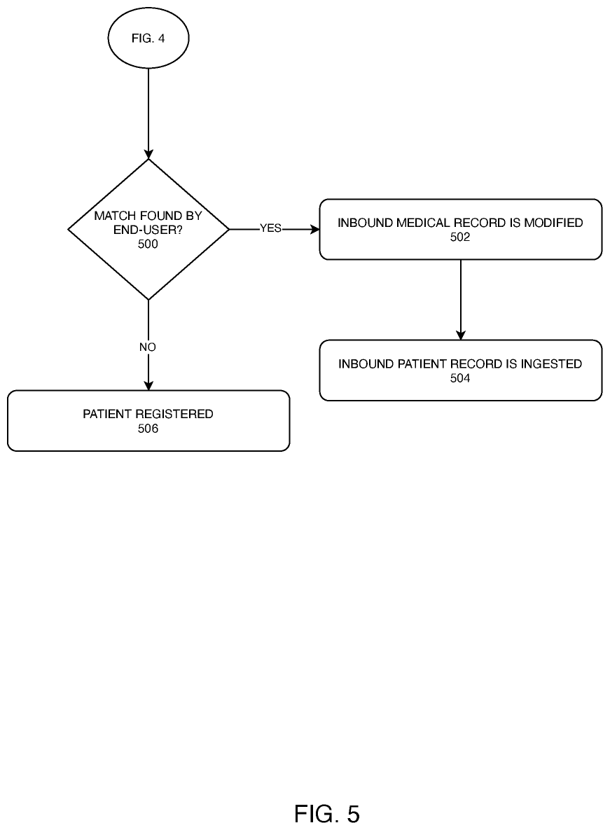

If the “send to checkpoint and retry” option is determined not been enabled in step 404, then the process continues to step 416 where the inbound medical record passed to the Checkpoint and the requesting medical provider 100 is prompted to perform a manual review of the inbound medical record. In an embodiment, no further routing or action is taken on the inbound medical record until manual intervention occurs by the requesting medical provider 100. The process then continues to step 500, as is described in more detail herein with regards to FIG. 5.

In step 408, the virtual service 114 determines if a retry attempt is permitted based on comparing, for example, a counter to the retry attempt criteria. In an embodiment, the counter can be compared to a maximum number of retries that may be attempted, where the counter is incremented with each retry attempt performed by the virtual service 114 on the inbound medical record. If the virtual service 114 determines that a retry attempt is permitted in step 408, then the virtual service 114 in step 410 determines if a wait time has elapsed since a previous patient matching attempt. If the wait time has elapsed, then the process continued to step 300 in FIG. 3, as described with more detail herein.

If, however, the counter exceeds a maximum number of retries specified by the retry attempt criteria, then the process continues to step 412 where the virtual service 114 determines if a “send to routing rules” option has been enabled by the administrator. If the “send to routing rules” option has been enabled, then in step 414, the inbound medical record is passed to medical provider 100 based on the pre-defined routing rules. In an embodiment, the inbound medical record is modified with a configured identifier and ingested for review by the medical provider 100. The inbound medical record can then be manually reconciled by the medical provider 100, and then ingested by the medical provider 100 into its database 108, and an in embodiment, specifically into a DICOM storage or archive.

FIG. 5 is a flowchart illustrating the steps of ingesting an inbound medical record based on a manual review, according to an embodiment of the invention. In step 500, an end-user of the medical provider 100 performs a manual review of the database 108 to determine if there are any medical records stored in the database 108 which match the patient associated with the inbound medical record. If no matching medical record is identified in step 500, then the medical network operator 105 (via the virtual service 114) registers a patient based on the inbound medical record. In another embodiment, if no matching record is identified in step 500, then the inbound medical record remains in the Checkpoint for a subsequent manual review, is ingested into the database 108, or is rejected by the medical provider 100.

If, however, in step 500, a matching medical record is identified in the database 108, then in step 502 the patient demographic information of the inbound medical record is modified. In an embodiment, at least one patient identifier in the inbound medical record is modified so that it is consistent with the corresponding patient identifier in the matching medical record. For example, in an embodiment, the virtual service 114 can update the MRN on the inbound medical record to match the MRN on the matching medical record. In many instances, the medical provider 100 may have an MRN numbering and/or classification scheme that is not consistent with MRN scheme used by the external medical providers 102.

Next in step 504, the modified inbound medical record is then ingested by the medical provider 100 into its database 108, and an in embodiment, specifically into a DICOM storage or archive. In another embodiment, the modified inbound medical record is ingested into a patient scheduling system or patient record system. In an embodiment, the modified inbound medical record is transmitted from the virtual service 114 to a DICOM archive or storage associated with the database 108.

In an embodiment, an audit entry can also be recorded with the storage location of the match and final storage locations. The audit entry can include, for example, the modifications or transformations undertaken on the inbound medical record, and whether such modifications or transformations were performed automatically, or were performed manually by the medical provider 100. In an embodiment, each attempted patient matching process is logged in an audit trail. Whenever a match occurs, whether manually, or automatically by the virtual service 114, an audit entry is recorded which shows the inbound medical record was matched to a patient with medical records stored by the medical provider 100, the attributed of the inbound medical record that were modified, the DICOM storage location of the matching record, and the DICOM storage location for the modified inbound medical record.

In an embodiment, the audit entry can be stored on database 112 or local database 800, can be accessible by the medical network operator 105, as well as the medical provider 100, and the external medical provider 102 which transmitted the inbound medical record. In another embodiment, the audit entry may only be accessible to the medical provider 100, or only be accessible to the medical network operator 105.

FIG. 6 is a flowchart illustrating the steps of creating a patient matching configuration file, according to an embodiment of the invention. In step 600, an administrator can access a portal, such as a secure website via a Uniform Resource Location (URL) using a browser on a computing device. In an embodiment, the computing device can be a PACS station, a medical workstation, and the like.

The portal can be stored on, or executed from, for example, the medical network 104, and the portal allows the administrator to configure various settings and criteria for the automated patient matching function.

In an embodiment, prior to being able to access the portal, the administrator must enter credentials, such as a login and password, or other indicia that verifies their identity. The credentials can include user's mobile device number, login, password, e-mail address, phone number, account number, personal identification number (PIN), name, driver's license number, social security number, birthdate, employee number, and/or a unique account identification code previously provided to the administrator by an authorizing entity, such as an employer or the medical network operator 105. In another embodiment, the credentials can be biometric, such as a fingerprint, iris, facial, or voice scan. In yet another embodiment, the credential can be a gesture input by the user, such as a on a touchscreen or touchpad.

In an embodiment, the portal can be accessed using a federated login, a Security Assertion Markup Language (SAML) function, an Active Directory (AD) login, and the like. In addition, the portal can utilize various authentication mechanisms, such as, but not limited to, HTTP basic authentication, form-based login authentication, client-certificate authentication, mutual authentication, digest authentication, and the like.

In an embodiment, the administrator is affiliated with the medical provider 100, and can be an information technology professional, an end-user, medical record keeping personnel, medical office or practice manager, PACS administrator, a physician, a nurse, and the like.

In an embodiment, the administrator can be affiliated with the medical network operator 105. In this embodiment, the medical provider 100 and medical network operator 105 can have a business arrangement or contractual agreement in place that allows the administrator to access the portal on behalf of the medical provider 100. In this embodiment, the administrator may remotely access the portal via the virtual service 114.

In step 602, the administrator selects various action options to take based on a patient matching outcome, for example, if there is a single match, no match, multiple matches, etc. In an embodiment, the action options can include, for example, sending the inbound medical record based on pre-defined routing rules (i.e., “send to routing rules”), sending the inbound medical record to the Checkpoint (i.e., “send to checkpoint”), sending the inbound medical record to the patient matching function and retrying the patient matching process (i.e., “send to checkpoint and retry”), and the like.

In an embodiment, the action options can be specified for various patient matching outcomes. For an action on a single match, the setting allows the administrator to “send to routing rules”, or “send to the patient matching function”. For an action on multiple matches or no matches, the setting allows the administrator to “send to routing rules”, “send to the patient matching function” or “send to the patient matching function and retry”.

In step 604, if the virtual service 114 determines if “send to routing rules” option is selected. If the “send to routing rules” option is selected, then in step 606, the administrator can specify or define the routing location, which can be stored in a routing rules configuration file. In an embodiment, the DICOM and HL7 locations are defined so that if inbound medical records are successfully matched to a patient with medical records stored by the medical provider 100 (i.e., stored in database 108), then the virtual service 114 has a route for where to send modified inbound medical records.

If, however, the “send to routing rules” option is not selected, then in step 608, the virtual service 114 determines if the “send to the patient matching function and retry” option has been selected by the administrator. If the “send to the patient matching function and retry” option has been selected, then in step 610, the administrator specifies retry attempt values. In an embodiment, the retry attempt values can include, for example, a retry frequency, maximum number of retries that may be attempted, and/or a wait time between retry attempts. In an embodiment, the retry attempt values can include a maximum number of retries that may be attempted within a predefined time period. For example, the administrator may limit the retry attempts to five attempts in a 24-hour period, to two attempts in per hour, etc. These examples are non-limiting illustrations, and not intended to limit the scope of the invention.

In an embodiment, during a matching retry process, the inbound medical records are held in the Checkpoint so that the medical provider 100 can elect to manually reconcile the inbound medical record. The patient matching retry process can be applied when multiple matches are identified, however, the retry process is not executed when a single match is obtained. For multiple matches, the patient matching retry process allows the system to obtain new information from the medical provider 100, such as via a patient record system or patient scheduling system.

In an embodiment, when either a no match or a multiple match condition occurs while the inbound medical record is in the Checkpoint, the administrator can modify the demographic data, manually query the database 108, or select from a list of candidate matches identified by the virtual service 114. If the administrator modifies the demographic data on the inbound medical record, the modifications can be reviewed and confirmed prior to acceptance and ingesting the modified inbound medical record by the medical provider 100.

If, however, the “send to the patient matching function and retry” option is not selected, then the virtual service 114 determines that the “send to the patient matching function” option is selected in step 612. The “send to the patient matching function” option will result in the inbound medical records being held in the Checkpoint for manual review by an end user. In step 614, the administrator defines exclusion rules that would result in inbound medical records bypassing the patient matching process. In an embodiment, the exclusion rules are a distinct set of rules that are different from the routing rules. In an embodiment, the routing rules can incorporate the exclusion rules. In a preferred embodiment, the exclusion rules are stored in an exclusion rules file.

In step 616, the administrator specifies whether patient identifiers and/or MRNs are standardized across all storage locations associated with the medical provider 100. In an embodiment, the virtual service 114 can reject matches where a matched patient record location is not the same as the destination storage location (i.e., the located patient information is stored in a storage location that does not match the destination storage location defined in the routing rules).

In step 618, the administrator specifies whether a patient middle name should be used query the database 108 during the patient matching process. For example, if the medical provider 100 does not store a patient's middle name in their database 108, either by choice, preference or technical limitation, then this setting allows the patient matching process to disregard a patient's middle name or middle initial when analyzing inbound medical records. In an embodiment, if a patient middle name is not utilized for the patient matching process, then this setting can inherently decrease the uniqueness of candidate matches due to the exclusion of the middle name attribute.

In step 620, the administrator specifies if a non-numeric patient identifier and/or MRN in the inbound medical record should be disregarded. In an embodiment, a non-numeric patient identifier may indicate that the inbound medical record was not properly reconciled by the external medical provider 102.

In step 622, the patient matching configuration file is generated and stored by the virtual service 114 based on the settings and options selected in steps 602-620 described herein. In an embodiment, the virtual service 114 generates the patient matching configuration file, and the patient matching configuration file is uploaded to the database 112 coupled to the medical network 104. In another embodiment, the patient matching configuration file is stored on the local database 800 coupled to the virtual service 114. The configuration file can be retrieved at a later time by the administrator (or another administrator) in order to duplicate or modify the automated patient matching settings. In an embodiment, the patient matching configuration file is in a comma-separated values (CSV) format, a JSON format, an XML format, a text format, and the like.

In an embodiment, the routing rules configuration file and the exclusion rules file can be generated and stored in a similar fashion as the patient matching configuration file.

Below are exemplary patient matching queries and outcomes, according to embodiments of the invention.

TABLE 1

| Inbound Patient | Patient Name in | Date of | | Patient |
|---|---|---|---|---|
| Name | the Database | Birth | Sex | ID |
| Bob Smith | Bob Smith | 1965 Jan. 01 | M | 1234 |
| | Bob Ryan Smith | 1965 Jan. 01 | M | 2345 |
| | Bob R Smith | 1965 Jan. 01 | M | 2345 |

In Table 1, the inbound patient attributes for “Bob Smith” return three results from the database 108. Two of the three results contain the same patient identifier; thus, these two results are potentially the same patient. However, since there are multiple distinct records resulting from the query, the virtual service 114 fails to automatically match the inbound medical record and sends the results into the patient matching function for manual review.

TABLE 2

| Inbound | Patient Name in | Date of | | |
|---|---|---|---|---|
| Patient Name | the Database | Birth | Sex | Patient ID |
| Bob Smith | Bob Smith | 1965 Jan. 01 | M | 1234 |

In Table 2, the inbound patient attributes for “Bob Smith” returns a single result. Since there is a single potential matching record, the virtual service 114 automatically matches the inbound medical record and updates the inbound medical record with the specified attributes contained in the database 108.

TABLE 3

| Inbound | Patient Name in | Date of | | |
|---|---|---|---|---|
| Patient Name | the Database | Birth | Sex | Patient ID |
| Bob Smith | Bob Smith | 1965 Jan. 01 | M | 1234 |
| | Bob Ryan Smith | 1965 Jan. 01 | M | 2345 |

In Table 3, the inbound patient attributes for “Bob Smith” returns two results. While the date of birth and the sex of the two resulting records are the same, the patient name in one record contains a middle name and the patient identifier for each resulting record is different. Since the inbound medical record does not contain a middle name or middle initial, and because the patient identifiers are different, the virtual service 114 fails to automatically match the inbound medical record and sends the two potential results to the patient matching function for manual review.

TABLE 4

| Inbound | Patient Name in | Date of | | |
|---|---|---|---|---|
| Patient Name | the Database | Birth | Sex | Patient ID |
| Bob Smith | Bob Smith | 1965 Jan. 01 | M | 1234 |
| | Bob Ryan Smith | 1965 Jan. 01 | M | 1234 |

In Table 4, the inbound patient attributes for “Bob Smith” returns two results. While the date of birth and the sex of the two resulting records are the same, the patient name in one record contains a middle name. Since the inbound medical record does not contain a middle name or middle initial, virtual service 114 fails to automatically match the inbound medical record and sends the two potential results to the patient matching function for manual review.

In another embodiment, the administrator can configure the matching criteria, such that in the scenarios shown in Table 3 and Table 4, the virtual service 114 can identifies a match.

TABLE 5

| Inbound | Patient Name in | Date of | | |
|---|---|---|---|---|
| Patient Name | the Database | Birth | Sex | Patient ID |
| Bob R Smith | Bob R Smith | 1965 Jan. 01 | M | 1234 |

In Table 5, the inbound patient attributes for “Bob R Smith” returns a single result. Since there is a single potential matching record, the virtual service 114 automatically matches the inbound medical record and updates the inbound medical record with the specified attributes contained in the database 108.

TABLE 6

| Inbound | Patient Name in | Date of | | |
|---|---|---|---|---|
| Patient Name | the Database | Birth | Sex | Patient ID |
| Bob Ryan Smith | Bob Ryan Smith | 1965 Jan. 01 | M | 1234 |

In Table 6, the inbound patient attributes for “Bob Ryan Smith” returns a single result since the inbound patient first, middle and last name match the same fields in the resulting record in the database 108. Since there is a single potential matching record, the virtual service 114 automatically matches the inbound medical record and updates the inbound medical record with the specified attributes contained in the database 108.

TABLE 7

| Inbound | Patient Name in | Date of | | |
|---|---|---|---|---|
| Patient Name | the Database | Birth | Sex | Patient ID |
| Robert R Smith | Bob R Smith | 1965 Jan. 01 | M | 1234 |

In Table 7, the inbound patient attributes for “Robert R Smith” would not return any results from the patient matching query. Thus, the virtual service 114 fails to automatically match the inbound medical record and sends the inbound medical record to the patient matching function for manual review.

TABLE 8

| Inbound | Patient Name in | Date of | | |
|---|---|---|---|---|
| Patient Name | the Database | Birth | Sex | Patient ID |
| Maria Garcia | Maria Garcia- | 1965 Jan. 01 | F | 1234 |
| | Hernandez | | | |
| | Marcia Garcia | 1965 Jan. 01 | F | 2345 |

In Table 8, the inbound patient attributes for “Maria Garcia” returns two results. Thus, the virtual service 114 fails to automatically match the inbound medical record and sends the inbound medical record to the patient matching function for manual review as the patient name and patient ID are different for the two resulting records. The two records have the same date of birth and gender; however, the patient name contains a hyphenated last name and the patient identifiers are different. Since the two results have different patient identifiers, the virtual service 114 fails to automatically match the inbound medical record and sends the two potential results to the patient matching function for manual review.

In an embodiment, the virtual service 114 can utilize artificial intelligence matching, such as fuzzy matching or stochastic matching, to determine that a patient got married, divorced, had a name change (first, last, and/or middle), had a sex change, address change, insurance change, and the like, and would then use this determination in the matching process. For example, in the scenario depicted in Table 8, the matching technique could determine that the individual named “Maria Garcia” was previously unmarried, and then got married, and would infer that the record for “Maria Garcia-Hernandez” is for the same individual that now includes her married name.

In an embodiment, the patient matching function can utilize fields in addition to the sex, first name, last name, middle name, and date of birth. In an embodiment, medical information such as a medical history, clinical data, procedure or treatment history, prescription history, and the like can, be analyzed using artificial intelligence matching. In an example, if a medical history for a potential matching record does fully match the medical history of an inbound medical record (i.e., certain encounters, orders, visits, and/or procedures are missing, etc.), however, other fields do match, then artificial intelligence matching can determine that a potential match does in fact exist.

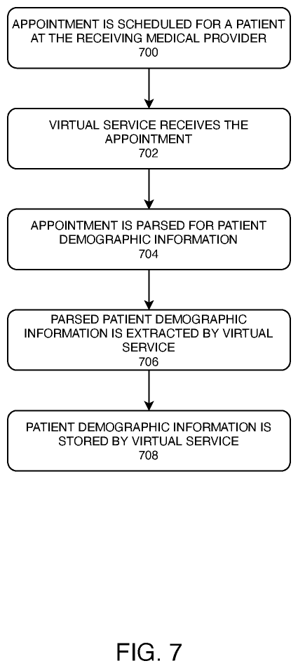

FIG. 7 is a flowchart illustrating the steps of retrieving patient demographic information from an appointment order for an advanced patient matching process, according to an embodiment of the invention. In step 700, the medical provider 100 enters an appointment for a patient. The appointment can include, for example, patient demographic information, appointment details, and/or procedure identifiers.

In an embodiment, the patient demographic information can include, but are not limited to, a patient first, middle, and/or last name, date of birth, social security number, patient identifiers, sex, address, telephone number, insurance information, primary care physician information, universal identifier, accession number, medical record number, and the like.

In an embodiment, the appointment details can include, but is not limited to an appointment date, appointment time, appointment facility/location, STAT or RUSH order instructions, etc., as well as prescribing physician and referred physician information, pre-appointment instructions, etc.

In an embodiment, the medical provider 100 enters a patient appointment into a patient scheduling system 118 that is integrated with the EHR system via a portal (i.e., such as in a FHIR compatible EHR or in an EHR with API access) In an embodiment, the patient appointment can be generated remotely and electronically entered or pushed into the patient scheduling system 118, which can integrated into, or coupled with, for example, an EHR, a physician referral system, a laboratory testing ordering system, and the like.

In step 702, a message related to the patient appointment is transmitted to the virtual service 114 by the patient scheduling system 118. In an embodiment, the message can include the appointment details, procedure identifiers, and patient identifiers. In an embodiment, the virtual service 114 is configured to receive push notifications or messages from the patient scheduling system 118 in real-time. In another embodiment, the virtual service 114 is configured to receive push notifications or messages from the patient scheduling system 118 periodically, such as based on a pre-defined interval of every minute, every thirty minutes, every hour, once daily, once weekly, and the like. The pre-defined interval can be selected by the requesting medical provider 100, and can be dynamically adjusted based on preference, patient volumes, medical facility type (i.e. critical care, emergency, and the like may require more frequent pushes versus a long-term care, recovery and rehabilitation facilities, and the like).

In another embodiment, in step 702, the virtual service 114 listens to, and analyzes, a data feed from a patient scheduling system 118 to determine if a patient appointment has been entered into the patient scheduling system 118. In an embodiment, the virtual service 114 is communicatively coupled to the patient scheduling system 118 via a data exchange interface. Such connectivity allows the virtual service 114 to access, receive data from, or subscribe to, a data feed from the patient scheduling system 118. The virtual service 114 can “listen” to the data feed, and analyze and monitor the data feed for various information. The data feed can provide various information to the virtual service 114, including, but not limited to, patient appointments entered into, or received by, the patient scheduling system 118.

In an embodiment, the virtual service 114 is configured to actively receive the data feed from the patient scheduling system 118 whenever a change, update, or access to the patient scheduling system 118 occurs. In another embodiment, the virtual service 114 is configured to receive the data feed periodically, such as based on a pre-defined interval of every minute, every thirty minutes, every hour, once daily, once weekly, and the like. The pre-defined interval can be selected by the requesting medical provider 100, and can be dynamically adjusted based on preference, patient volumes, medical facility type (i.e. critical care, emergency, and the like may require more frequent data feed pushes versus a long-term care, recovery and rehabilitation facilities, and the like).

In another embodiment, the requesting medical provider 100 can manually request the data feed to be transmitted to the virtual service 114. In another embodiment, information related to the patient appointment can be transmitted directly to the virtual service 114, instead of the virtual service 114 listening to the data feed and analyzing the data feed to identify a patient appointment.

Next in step 704, the virtual service 114 parses the data feed to identify patient demographic information from the patient appointment order. In an embodiment, the virtual service 114 includes parsing engine to analyze text, characters, numbers, and alphanumeric contents located within the data feed.

In an embodiment, the data feed may be in any data exchange format as described herein. In a preferred embodiment, the data feed is in a HL7 format. The parsing engine may be configured to parse the data feed in the HL7 format, and may be configured to assign a dictionary to the data feed in the HL7 format, so that the data feed may be translated. The parser 116 may be configured to parse data that is formatted in a HPRIM, GMSIH, JAHIS format, or any other format suitable and compliant with IHE standards. In addition, parsing engines dedicated to the different versions of HL7 as well as various other medical data standards and/or dedicated to other known formats may be utilized by the parsing engine without departing from the scope of the present invention. In an embodiment, the parsing engine can utilize XML parsing, text-based parsing, and the like.

In an embodiment, the virtual service 114 may include one or more parsing engines, whereby a first parsing engine may be configured to parse any incoming information and/or distribute the incoming data to other parsing engines.

In an embodiment, the parsing engine can utilize a machine learning function or algorithm to parse the inbound medical record, as well as to contribute to a dynamically updated learning dictionary. By using machine learning analysis on information extracted from data feeds over time, the parsing engine can be optimized to predictively optimize text and character recognition and analysis functions.

After parsing, the virtual service 114 extracts the patient demographic information from the patient appointment order in step 706. In an embodiment, the extracted information can further include a mapping location extracted from the data feed related to the patient appointment. The mapping location can include, but is not limited to, a storage location, a network location or space, a database location, and the like that is mapped to an attribute in the patient appointment.

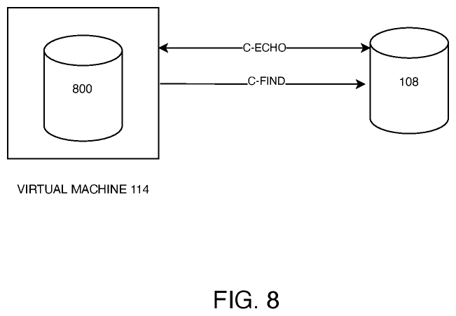

Next, in step 708, the extracted patient demographic information is stored on the local database 800 coupled to the virtual service. FIG. 8 depicts the local database 800. In an embodiment, the local database can be database 112 coupled to the medical network 104.

In an embodiment, the advanced patient matching process utilizes the extracted patient demographic information for comparison against the inbound medical record. In this embodiment, instead of comparing the metadata of the inbound medical record against a storage location or multiple storage locations associated with the medical provider 100, the virtual service 114 compares the inbound medical record against the extracted patient demographic information.

In an embodiment, a patient identifier from an appointment order generated by the medical provider 100 is recorded along with patient demographic attributes that were associated with DICOM-based deterministic patient matching. The patient identifier is recorded to account for potential changes to a patient's medical record. An example of a change that may occur is a last name change following a marriage or a divorce. In this scenario, the virtual service 114 compares the medical records containing duplicate patient identifiers and evaluates if the patient demographic information differs. If the patient demographic information differs, a duplicate entry is permitted to be written to the database 108. The duplicate entry is permitted as the medical provider 100 may receive inbound medical records for a patient that uses the patient's previous last name. With a duplicate entry, the virtual service 114 can still match the inbound medical record and use logic to update the inbound medical record to match the stored patient demographic information associated with the same patient identifier.

Alternatively, it may be possible to write an alternate name attribute to a new or existing medical record in the database 108 that can be used for comparison purposes. If the patient demographic information matches, the database consequently does not need to be updated.

In the advanced patient matching process, a message, such as a HL7 Order Entry Message (OE or ORM), is generated by the medical provider 100, and the virtual service 114 receives the message, parses the message, and stores the patient demographic information in the local database coupled to the virtual service. This behavior is independent of whether or not the medical provider 100 is actually receiving prior imaging for a patient.

In an embodiment, when inbound medical records are transmitted to the medical provider 100 via the medical network 104 using the EHR/HL7 communication, the virtual service 114 can first attempt to query the local database containing the stored extracted patient demographic information parsed from the appointment order (i.e., parsed from the ORM message contents). If no matches are identified, the virtual service 114 falls back to querying the database 108, as described herein with more detail with reference to step 206 of FIG. 2.

In an embodiment, the virtual service 114 can attempt to deterministically match the patient demographic information in the inbound medical record with matches resulting from the patient matching query transmitted to the database 108. If this step fails to locate a match, then the inbound medical record is flagged for review and sent to the Checkpoint.

If, however, a match is obtained against the local database or database 108, the virtual service 114 can transmit an unsolicited ORU result with the updated patient demographic information. In an embodiment, the virtual service 114 can generate an unsolicited HL7 Observation Result (ORU) message or ORM with an accession number. The accession number can be generated by the virtual service 114 and/or the medical network 104. The accession number can be a unique identifier used by the medical network 104 to track, search, store, etc. prior medical records that have been accessed via the automated patient matching functions described herein, and the accession number is not necessarily linked to any local identification scheme utilized by any medical providers 100, 102.

In another embodiment, the accession number is passed to the medical provider 100, which can then return an internal accession number generated and/or utilized by the medical provider 100 and its associated patient record system. The virtual service 114 receives the internal accession number, and updates the accession number accordingly to match.

The unsolicited ORU result can contain an accession number associated with the medical network operator 105 and/or the virtual service 114. In this embodiment, the medical provider 100 can then generate an ORM message that is pushed to the database 108 (i.e., for example, pushed to a PACS and logged in an EHR).

Once the ORM has been generated and pushed to the database 108, the virtual service 114 sends the inbound medical record to the database 108 with the correct or updated patient demographic information and the same accession number used in the unsolicited ORU result.

In an embodiment, to increase the likelihood of a successful match of the inbound medical record patient demographic attributes to the attributes stored by the medical provider 100, the virtual service 114 can pre-process and/or homogenize the inbound medical record. Such pre-processing may not be required and can be configured by the administrator such that inbound medical records can be queried “as-is”, or can be queried after pre-processing.

In an embodiment, the pre-processing can be performed by a software instance executing on the virtual service 114, or a hardware processor coupled to the virtual service 114.

In an embodiment, if pre-processing is performed, the following processing steps can be executed by the virtual service 114 on the patient demographic information of the inbound medical record: removal of spaces, removal of dashes and hyphens, removal of periods and other punctuation, homogenizing of capitalization (i.e., all upper case or all lower case) to match a format specified in the patient matching configuration file, homogenizing of formatting of the date attributes to match a format specified in the patient matching configuration file (i.e., YYYMMDD, MMDDYYYY, etc.), and/or homogenizing of formatting of sex attributes to match a format specified in the patient matching configuration file (e.g. “Female” or “F”).

In an embodiment, the advanced patient matching process can utilize a string comparison algorithm to generate similarity scores when comparing patient demographics in the inbound medical record and the local medical records stored in the databased 102. The patient demographics can include, for example, a patient name. In an embodiment, a k-means clustering method is utilized by the virtual service 114 for classification of results.

FIG. 8 is a block diagram illustrating a local database coupled to a virtual service, according to an embodiment of the invention. In an embodiment, the virtual service 114 includes a local database 800 coupled to the virtual service 114. The local database 800 can reside within the virtual service 114, or can be located remotely, such as integrated with database 112. In another embodiment, the database 112 is utilized instead of the local database 800. In an embodiment, the local database 800 is a cross-platform document orientated database, such as MongoDB.

In an embodiment, the virtual service 114 coupled to the database 108 that contains medical records associated with the medical provider 100. In order to issue a patient matching query, the virtual service 114 transmits a C-ECHO request to the database 108. In an embodiment, the database 108 relays the C-ECHO request to database 108 to determine if database 108 is online.

If the database 108 is online, the virtual service 114 transmits a C-FIND request to the database 108 to determine if there are any medical records stored in the database 108 which match the patient associated with the inbound medical record.

In an embodiment, the functions of the virtual service 114 can be executed by the medical network server 122. It is understood that any of the external medical providers 102 can also function as a receiving medical provider 100, and the receiving medical provider can function as an external medical provider.

In yet another embodiment, the patient scheduling systems 118, 120 can perform the functions of the virtual service 114, 116, and can operate an instance of the medical network server 122. In this embodiment where the patient scheduling systems 118, 120 are EHRs, each EHR can directly communicate with another EHR (i.e., direct EHR-to-EHR communication), where medical records can be transferred directly between EHRs located at disparate medical providers.

In an embodiment, the virtual service 114 can leverage a machine learning function or algorithm in order to analyze various data that is processed by the virtual service 114 and/or the medical network 104. For example, information extracted from inbound medical records over time, such as patient demographic information and patient identifiers, can be stored in the local database 800. Similarly, information regarding inconsistencies in patient demographic information between external medical records and local medical records can be stored in the local database 80. Such information can be analyzed over time by the machine learning function to identify trends, patterns, and insights that can be leveraged by the virtual service 114 to predictively optimize the processes described herein for automated patient matching.

In an embodiment, output generated from the string comparison algorithm can be used as an input to the machine learning function in order to predictively optimize the string comparison algorithm over time.

While the principles of the disclosure have been illustrated in relation to the exemplary embodiments shown herein, the principles of the disclosure are not limited thereto and include any modification, variation or permutation thereof.

What is claimed is:

1. A method for automated matching of disparate medical records for a patient, comprising:
receiving appointment information for the patient by a server;
extracting, by the server, a patient attribute and a medical record identifier from the appointment information;
storing the patient attribute in a database coupled to the server;
receiving, by the server, an inbound medical record, wherein the inbound medical record contains an inbound patient attribute and an inbound medical record identifier; and
determining, by the server, if the inbound patient attribute matches the patient attribute,
wherein the server is configured to update the inbound medical record identifier to match the medical record identifier if the inbound patient attribute matches the patient attribute.

2. The method of claim 1, wherein the patient attribute is selected from a group consisting of a first name, a middle name, a middle initial, a last name, a date of birth, and a gender.

3. The method of claim 1, wherein the medical record identifier is selected from a group consisting of a universal identifier, an accession number, a medical record number, an identification number, a patient number, a chart number, and a series number.

4. The method of claim 1, wherein the server is a virtual service operating as a virtual instance of a networked server.

5. The method of claim 1, wherein the server receives the appointment information from a patient scheduling system that is communicatively coupled to the server via a data exchange interface.

6. The method of claim 1, further comprising parsing, by the server, the appointment information using a parsing engine.

7. The method of claim 1, wherein the appointment information is in in a format selected from a group consisting of the Health Level Seven (HL7) standard, the Fast Healthcare Interoperability Resources (FHIR) standard, the HPRIM standard, the Continuity of Card Record (CCR) standard, the Continuity of Care Document (CCD) specification, the xDT data exchange format, and the Arden syntax.

8. A method for automated matching of disparate medical records, comprising:
receiving, by a server, an inbound medical record from a remote database, wherein the inbound medical record includes demographic data and an identification number;
comparing, by the server, the demographic data in the inbound medical record to stored demographic data in a stored medical record located on a local database, wherein the stored medical record includes a stored medical record identification number, and wherein the local database is communicatively coupled to the server; and
updating, by the server, the identification number to match the stored medical record identification number if the demographic data matches the stored demographic data.

9. The method of claim 8, further comprising, requiring, by the server, a manual review of the inbound medical record if the demographic data does not match the stored demographic data.

10. The method of claim 8, wherein the inbound medical record is a medical imaging study.

11. The method of claim 8, wherein the local database is part of a patient record system.

12. The method of claim 8, wherein the demographic information is selected from a group consisting of a first name, a middle name, a middle initial, a last name, a date of birth, and a gender.

13. The method of claim 8, wherein the identification number is selected from a group consisting of a universal identifier, an accession number, a medical record number, an identification number, a patient number, a chart number, and a series number.

14. A system for automated matching of disparate medical records, comprising:
a server coupled to a medical provider;
a virtual service coupled to the medical provider, wherein the virtual service is an instance of the server;
a patient scheduling system coupled to the virtual service via a data exchange interface, wherein the virtual service is configured to receive appointment information from the patient scheduling system;
a parsing engine coupled to the virtual service, wherein the virtual service is configured to extract patient demographic information from the appointment information using the parsing engine; and
a database coupled to the virtual service, wherein the virtual service is configured to store the extracted patient demographic information on the database,
wherein the virtual service is further configured to compare an inbound medical record to the extracted patient demographic information.

15. The system of claim 14, wherein the data exchange interface utilizes a format selected from a group consisting of the Health Level Seven (HL7) standard, the Fast Healthcare Interoperability Resources (FHIR) standard, the HPRIM standard, the Continuity of Card Record (CCR) standard, the Continuity of Care Document (CCD) specification, the xDT data exchange format, and the Arden syntax.

16. The system of claim 14, wherein the extracted patient demographic information is selected from a group consisting of a first name, a middle name, a middle initial, a last name, a date of birth, and a gender.

17. The system of claim 14, wherein the virtual service is further configured to update an identifier on the inbound medical record if the inbound medical record matches the extracted patient demographic information.

18. The system of claim 14, wherein the virtual service is configured compare the inbound medical record to the extracted patient demographic information using a patient matching configuration file.

19. The system of claim 14, wherein the virtual service is configured to perform pre-processing on the inbound medical record.

20. The system of claim 19, wherein the virtual service compares the extracted patient demographic information to information in the inbound medical record.
